(19) United States
(12) Patent Application Publication
(10) Pub. No.: US 2013/0149352 A1
(43) Pub. Date: Jun. 13, 2013

(54) APPARATUS AND METHOD FOR THE TREATMENT OF ABNORMAL UTERINE BLEEDING

(71) Applicant: Steven D. Goedeke, Forest Lake, MN (US)

(72) Inventor: Steven D. Goedeke, Forest Lake, MN (US)

(21) Appl. No.: 13/711,739

(22) Filed: Dec. 12, 2012

Related U.S. Application Data
(60) Provisional application No. 61/569,986, filed on Dec. 13, 2011.
(60) Provisional application No. 61/569,978, filed on Dec. 13, 2011.

(51) Int. Cl. A61K 31/4174; A61K 9/00

(52) U.S. Cl. CPC A61K 31/4174; A61K 9/0036
USPC 424/400; 514/401

(57) ABSTRACT

Method and apparatus are disclosed for applying a therapeutic amount of a vasoconstrictor within the vaginal canal to control abnormal uterine bleeding. The abnormal bleeding can be due to excessive menstrual blood flow, bleeding from a surgical procedure, postpartum bleeding or any other acute or chronic condition. The vasoconstrictor includes topical agents such as an alpha-adrenergic agonist, for example oxymetazoline. The vasoconstrictor can be applied within the vaginal canal using any of many delivery apparatus. The vasoconstrictor can be included on a carrier member that is positioned in the vaginal canal and remains in place for a period of time, such as a tampon. In some embodiments, the carrier member can be a polymer ring or other shape that is inserted in the upper portion of the vaginal canal, such as the formix area.

### FIELD OF THE INVENTION

The present invention relates generally to the field of gynecological medicine. More particularly, the present invention relates to methods and apparatus for treatment of abnormal or heavy uterine bleeding by delivering a therapeutic amount of a non-systemic vasoconstrictor into the vaginal canal.

### BACKGROUND

Abnormal uterine bleeding (AUB) has a significant impact on the life of many women. According to Marret, AUB accounts for up to 20% of visits to the gynecologist yet the causes and mechanisms of such dysfunction are less than clear. Marret, H., “Clinical Practice Guidelines on Menorrhagia”, European Journal of Obstetrics & Gynecology and Reproductive Biology 152 (2010) 133-137. Further, AUB is a frequently cited indication for hysterectomy and accounts for as many as 25% of all hysterectomies. Another study listed AUB as the main presenting problem in at least half of all the hysterectomies reported. Liu, “Systematic Review”, Value in Health, Vol. 10, No. 3 (2007) 183-194.

Blood is supplied to the uterus and its endometrium by the ovarian and uterine arteries which enter the wall of the uterus and give rise to arcuate arteries in the myometrium. Radial arteries branch from the arcuate arteries. Basal arteries branch from the radial arteries and cross the mometrial endometrial junction and supply blood to the basal endometrium via the spiral arterioles. Spiral arterioles run toward the endometrial surface or functional layer of the endometrium. The spiral arterioles give rise to the capillaries which form a plexus in the subepithelium of the endometrium. As a woman goes through the pre-ovulatory phase of the cycle, the length of the spiral arterioles increase five-fold, leading to coiling. Pre-menstrually, the endometrium regresses, and the spiral arterioles continue to coil. Just before the start of menses, blood flow slows in the spiral arteries due to vasoconstriction which appears to be followed by dilation of the arterioles and the onset of bleeding.

Abnormal and normal uterine bleeding are defined in terms of the regularity, frequency, duration and volume of menstrual bleeding. Although terminology varies, one accepted standard based on volume is that abnormal uterine bleeding is the loss of greater than 80 ml. of blood per menstruation. Heavy menstrual bleeding can also be defined in terms of frequency and duration of menstruation.

There can be many causes of abnormal or heavy uterine bleeding, some uterine causes and some systemic causes. Uterine causes can include fibroids, endometrial polyps, endometriosis and pelvic inflammatory disease. Systemic causes can include coagulation disorders and clotting factor deficiencies as well as hypothyroidism. However, about half of women with abnormal or heavy uterine bleeding have no anatomical or endocrinological abnormality that can be detected.

Current treatments can include endocrine based approaches. For example, some patients show a reduction in bleeding when taking oral contraceptives. Further, a levonorgesterel-releasing intrauterine device for birth control has shown a reduction in menstrual blood flow in women who have the IUD device implanted. However, both of these treatments can have side effects.

Other treatments can include a hysterectomy and endometrial ablation. However, both of these approaches will prevent the possibility of future pregnancy. Further, a hysterectomy is a major surgery and loss of the organ will require future hormonal drug therapy with attendant side effects.

There is a need for an alternative treatment option for abnormal or heavy menstrual bleeding. In particular, a treatment that does not require surgery or hormone therapy is preferred. Further, for many, the option of future pregnancy should be maintained with use of this alternative treatment. Finally, the alternative treatment should be free of detrimental side effects or risk that are present with current endocrine based treatment approaches.

### BRIEF SUMMARY

The present disclosure is directed to the application of a therapeutic amount of a non-systemic vasoconstrictor within the vaginal canal. The vasoconstrictor can be a topical vasoconstrictor. Further, the vasoconstrictor can be non-hormonal and/or non-steroidal. This can include an alpha-adrenergic agonist which can activate both alpha-1 and -2 receptors. One drug can include oxymetazoline or OMZ. Application within the vaginal canal can include application to the walls of the canal, formix area or the cervix prior to and/or during menorrhagia, which can be defined as excessive blood loss, as for example loss of greater than 80 ml. per menstruation. Heavy uterine bleeding can also be defined as frequent or long duration menstruation. Alternatively, the therapeutic amount of non-systemic vasoconstrictor can be applied prior to menstruation in individuals having a history of excessive blood loss during menstruation. The therapeutic amount can also be applied in other acute incidents of excessive uterine bleeding, as in post-surgery or postpartum situations that may arise.

The therapeutic amount of a vasoconstrictor can be delivered within the vaginal canal in a water-based solution or saline solution. Other carriers can also be used, such as gels. The gels may regulate the rate of release of the vasoconstrictor to the vaginal wall and extend the period of treatment relative to a saline carrier. For example, the therapeutic amount may be dispensed by the gel over a 72 hour period which could be equivalent to three daily doses. A combination of instant and time release medication can also be administered so that an initial bolus of vasoconstrictor causes immediate reduction in bleeding when placed in the vaginal canal while the time release portion continues to maintain the control of bleeding over an extended period of time.

It is believed application of a topical vasoconstrictor within the vaginal canal will result in a higher concentration of the vasoconstrictor within the uterine tissue relative to the rest of the body due to the first pass effect. In one method of treatment, a therapeutic amount of a topical vasoconstrictor is applied within the vaginal canal. The therapeutic amount of topical vasoconstrictor can be about 0.75 mg. to about 3 mg. Further, the topical vasoconstrictor can be an alpha-adrenergic agonist such as oxymetazoline. The topical vasoconstrictor can be non-hormonal and non-steroidal. The topical vasoconstrictor can include a water-based carrier such as saline which dissolves the vasoconstrictor. A therapeutic amount of the solution can include about 1 cc. to about 5 cc. of solution having a concentration of about 0.02% to about 0.08% by weight of topical vasoconstrictor.

The vasoconstrictor can be administered into the vaginal canal using a delivery system. The delivery system can include any system that places the vasoconstrictor in the vaginal canal. For example a syringe could be used to inject the vasoconstrictor, however, the drug may not all be retained in the vaginal canal. Therefore, in some embodiments, the vasoconstrictor can be included in a carrier material that generally adheres to the surface of the vaginal canal, such as a tissue compatible gel or adhesive. Alternatively, the vasoconstrictor can be included on a carrier member that is positioned in the vaginal canal and remains in place for a period of time. For example, the carrier member can be a tampon that is changed as necessary depending on blood flow or alternatively, the carrier member can be a suppository that dissolves within the vaginal canal. In some embodiments, the carrier member can be a polymer ring or other shape that is inserted in the upper portion of the vaginal canal, such as the formix area. The ring can include the vasoconstrictor in a polymer carrier that slowly releases over a period of time, such as three days, after which the ring is removed. The carrier members can include various surface structures to help retain position within the vaginal canal, such as projections or other surface shapes and contours that increase friction.

The above summary of some embodiments is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic plan view of a vasoconstrictor carrier member in the form of an absorbent tampon having the vasoconstrictor dispersed throughout at least a portion thereof.

FIG. 2 is partial cross section of a vasoconstrictor carrier member that includes an absorbent tampon having a coating layer that carries and releases a vasoconstrictor.

FIG. 3 depicts a delivery device for the tampon of FIG. 1 which can include a sealed structure for maintaining the vasoconstrictor on the tampon prior to delivery;

FIG. 4 is a schematic cross sectional view of the delivery device of FIG. 3 depicting the tampon therein and also an alternative embodiment wherein the vasoconstrictor is carried in a suppository located in the distal tip area of the delivery device.

FIG. 5 is a schematic plan view of an alternative carrier member in the form of a ring that is placed in the vaginal canal.

FIG. 6 is partial cross sectional view of the carrier of FIG. 5 depicting the vasoconstrictor carried in a polymer layer on the outer surface of the ring carrier structure.

FIG. 7 is an alternative cross sectional view of a ring vasoconstrictor carrier member that includes a porous outer surface and the vasoconstrictor therein for elution over time through the porous outer surface.

FIG. 8 depicts an alternative ring carrier member having an undulating shape which can improve retention in the vaginal canal and increase the area of contact with the vaginal wall.

FIG. 9 is a schematic plan view of another alternative carrier member having an outer surface including projections to aid in retention and increase surface contact.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF A PREFERRED EMBODIMENT

### Nomenclature

10 Carrier Member
12 Tampon
14 Body Portion
16 String Portion
20 Coating Layer
22 Formix Area
24 Device
26 Barrel
28 Plunger
30 Suppository
50 Carrier Member
52 Ring
54 Coating
56 Inner Ring
60 Pores
62 Chamber
64 Peaks
66 Valleys

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The present disclosure is directed to methods and apparatus for treatment of abnormal uterine bleeding or any other undesirable uterine bleeding. Abnormal uterine bleeding (AUB) is generally defined in terms of the menstrual cycle, to include a blood loss of greater than 80 ml. in a menstrual cycle. It can also be defined in terms of frequency and duration of menstruation. Although the present disclosure is primarily directed to treatment of AUB and generally discussed in that context, the method and apparatus are also useful for treating any undesirable uterine bleeding related and/or unrelated to menstruation. For example, post-surgery bleeding that may be associated with treating a uterine fibroid, or post-partum bleeding can be present after delivery of a child, or bleeding could be due to blood thinners in a patient's system. Therefore, for purposes of this disclosure, AUB is considered to include the various causes of undesirable bleeding from the uterus.

The present disclosure is directed to the application of a therapeutic amount of a non-systemic vasoconstrictor within the vaginal canal. The vasoconstrictor can be non-hormonal and/or non-steroidal. The vasoconstrictor can be a topical vasoconstrictor. This can include an alpha-adrenergic agonist which can activate both alpha-1 and -2 receptors. One drug can include oxymetazoline or OMZ. Application inside the vaginal canal can include application to the vaginal wall over a portion of its length, application in the area of the formix and/or application in the area of or on the cervix.

The vasoconstrictor is used to normalize uterine bleeding, not to completely stop bleeding, as such what constitutes the application of a therapeutic amount of a topical vasoconstrictor inside the vaginal canal can vary from patient to patient and also with each menstrual cycle of that patient. In some patients the therapeutic amount of topical vasoconstrictor is about 0.75 mg. to about 3 mg. in each daily cumulative dose, whether applied in a single bolus, or over a period of hours. In others, the daily dose is about 1 mg. to about 2 mg. daily, while in others the dose is about 1.5 mg. daily. The vasoconstrictor may be dissolved in a water-based carrier, such as saline, and can include a therapeutic amount of about 1 cc. to about 5 cc. having a concentration of about 0.02% to about 0.08% of topical vasoconstrictor. Alternatively, the dose can be about 2 cc. to about 4 cc. of the same concentration.

The vasoconstrictor can be placed in a controlled release carrier that forms a pellet or suppository structure that can be inserted into the vaginal canal. The pellet can include enough vasoconstrictor to provide treatment through the bleeding of one menstrual period and be inserted at or just prior to that cycle. Alternatively, the pellet could include enough vasoconstrictor for multiple cycles and be slow released for several months, including release during the time between menstrual cycles. To avoid treatment during non-bleeding, the pellet could be designed with an active release mechanism that opens dose containing compartments within the pellet in response to a signal from a controller. This type of dose delivery would use a remotely burned or eroded window on a dosing compartment. Alternatively, the doses could be encapsulated in differing carriers, such as biodegradable polymers of differing breakdown characteristics so that dosing occurs approximately on a monthly cycle. Different polymers can be used, or the same polymer having more or less cross linking can be used to vary the time of release. With this embodiment, the entire pellet or suppository can be bioresorbable. The pellet or carrier can be shaped for retention in the vaginal canal for a desired period of time. For example, the carrier member can be a ring structure that is positioned and retained in the area of the formix, which also allows for easy removal when desired.

The vasoconstrictor can be applied within the vaginal canal using any of many delivery apparatus. Some representative embodiments of delivery systems are included herein. The delivery system can include any system that places the vasoconstrictor in the vaginal canal. For example a syringe could be used to inject the vasoconstrictor, however, the drug may not all be retained in the vaginal canal if simply injected as a liquid. Therefore, in some embodiments, the vasoconstrictor can be included in a carrier material that generally adheres to the surface of the vaginal canal, such as a tissue compatible gel or adhesive. Alternatively, the vasoconstrictor can be included on a carrier member that is positioned in the vaginal canal and remains in place for a period of time. For example, the carrier member can be a tampon that is changed as necessary depending on blood flow or alternatively, the carrier member can be a suppository that dissolves within the vaginal canal. In some embodiments, the carrier member can be a polymer ring or other shape that is inserted in the upper portion of the vaginal canal, such as the formix area. The ring can include the vasoconstrictor in a polymer carrier that slowly releases over a period of time such as three days after which the ring is removed. The carrier member can include alternative shapes and/or include various surface structures to help retain position within the vaginal canal, such as undulations in the surface or projections extending from surfaces that increase friction.

Multiple doses of a vasoconstrictor could be included in the devices of the present invention. For example, the components could include a reservoir of vasoconstrictor and a patient activated pump or delivery system that is in fluid communication with the vaginal canal. The reservoir and pump could be positioned in the formix area and left for several days during a menstrual cycle or several cycles. Alternatively, the pump can be exterior to the body and connected via catheter.

The treatment cycle can vary from patient to patient under various embodiments of the present disclosure. For example, a single daily dose to the vaginal canal over a period of three days during menstrual bleeding may be sufficient. In other patients and treatment cycles, multiple daily doses may be necessary to reduce or control bleeding. Yet in other patients or treatment cycles, a continuous slow release may best control bleeding. Combinations of these cycles may also be beneficial. For example, at the start of menstrual bleeding a single higher dose or bolus followed by smaller support doses may most effectively control bleeding. For example, doses can be controlled by timing of the changing of tampons wherein each new tampon would include an initial bolus of vasoconstrictor followed by a lower dose over time until the tampon is changed.

Other therapeutic agents can be included with the vasoconstrictor. For example, some systems can include cleansing of the vaginal canal in the area of vasoconstrictor delivery prior to depositing the vasoconstrictor in contact with the vaginal wall. This can include antiseptic and/or antibacterial agents. Further, a pain reliever, such as a non-steroidal anti-inflammatory drug could be included with the vasoconstrictor to provide local pain relief, such as relief from menstrual cramping. Alternatively, a cleansing flush of the vaginal canal may precede application of the vasoconstrictor to clean the vaginal wall surface and allow better contact with the vasoconstrictor.

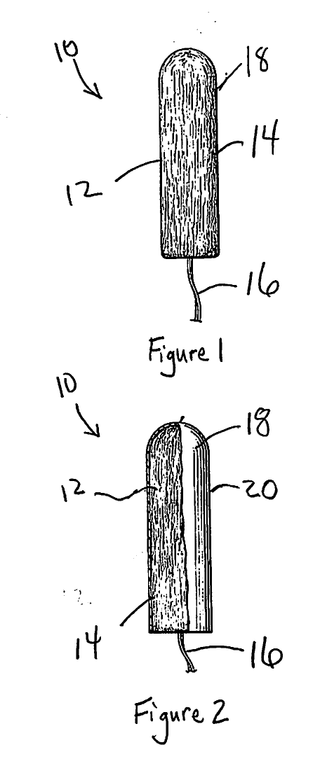

Now referring to FIG. 1, a representative carrier member 10 is depicted in the form of a tampon 12. In the embodiment depicted, the tampon 12 includes an absorbent body portion 14 and string portion 16 that extend external to the vaginal opening in use for ease of removing the tampon 12. The tampon 12 includes a therapeutic amount of a vasoconstrictor carried by the absorbent body portion 14. In particular, the vasoconstrictor can be on or embedded in the surface of the absorbent body portion 14. In some embodiments the vasoconstrictor is included on a distal portion 18 of the absorbent body portion 14 as it is believed placement in the upper portion of the vaginal canal will result in better transfer of vasoconstrictor to the uterine tissue.

As previously disclosed, the vasoconstrictor on the tampon 12 can be dissolved in a fluid carrier or may be mixed in a gel that retains it on the surface of the tampon 12 and places it in direct contact with the vaginal wall when the tampon 12 is inserted therein. The vasoconstrictor can also be a dry material that is wetted by the moisture within the vaginal canal to allow absorption by the tissue.

FIG. 2 depicts an alternative embodiment of a vasoconstrictor carrier member 10 in the form of a tampon 12 having coating layer 20 disposed over at least of portion of the surface of the tampon 12. In some embodiments, the coating layer 20 extends over only a distal portion of the absorbent body portion. The coating layer 20 has a vasoconstrictor dispersed therein. The coating layer 20 can be a soluble polymer that releases the vasoconstrictor over the time that the tampon 12 is deployed in the vaginal canal. In some embodiments the soluble polymer can form a sticky or gummy layer as it is wetted which causes adherence to the vaginal wall and aids in keeping the vasoconstrictor in contact with the tissue for absorption.

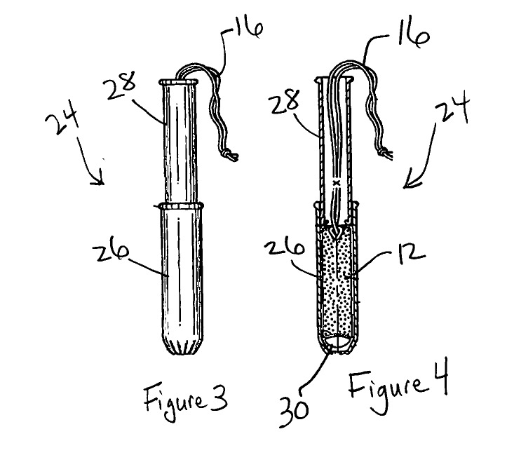

Now referring to FIG. 3, an insertion device 24 for deploying the tampon 12 is depicted. The insertion device 24 can include a barrel 26 having the tampon 12 compressed and positioned therein. A plunger 28 of smaller diameter is partially inserted into the barrel 26. Distal movement of the plunger 28 relative to the barrel 26 causes the tampon to exit the barrel 26 into the vaginal canal. FIG. 4 is a cross sectional view of an alternative embodiment, but also depicts the relationship between the plunger 28 and barrel 26 with the tampon 12 disposed therein.

In FIG. 4, the vasoconstrictor is included in a suppository 30 that is located in a distal portion of the barrel 26 adjacent the distal end of the tampon 12. With this embodiment, the vasoconstrictor can be delivered in conjunction with placement of the tampon 12 in the vaginal canal. After placement, the suppository 30 will be in a distal portion of the vaginal canal which can improve absorption into uterine tissue as it dissolves.

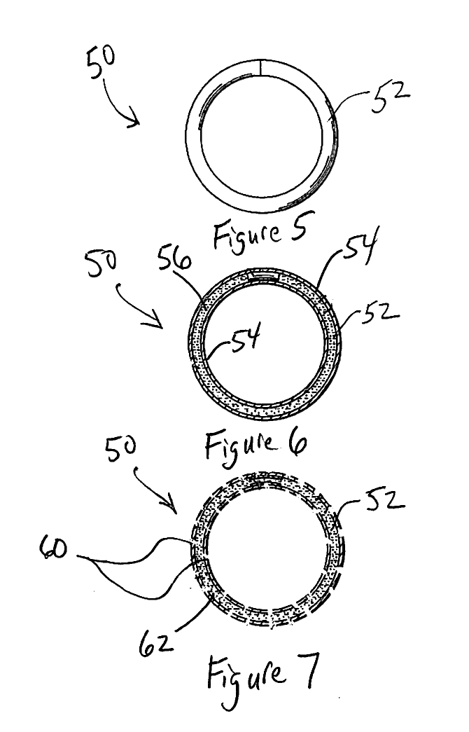

Now referring to FIG. 5, another alternative vasoconstrictor carrier member 50 is illustrated in the form of a ring 52. In this non-tampon embodiment any number of shapes can be utilized as are known in the art for pessary devices that are inserted into the vaginal canal and retained for a period of time. However, a ring structure can be used to fit comfortably in the formix area of the distal vaginal canal and may provide better transfer of vasoconstrictor into uterine tissue by virtue of its shape and position. In some embodiments the ring 52 or pessary dissolves over time and releases vasoconstrictor that is distributed throughout the soluble or resorbable device.

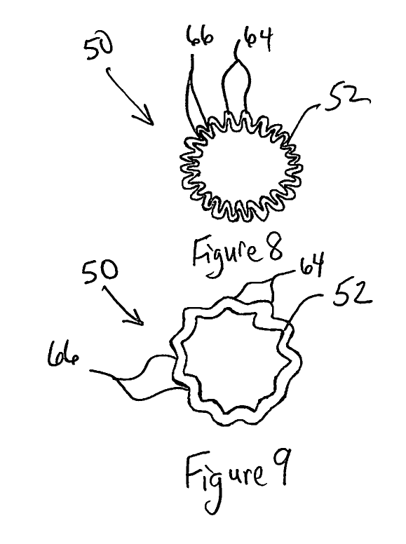

Alternatively, the ring 52 can include the vasoconstrictor within a coating 54 on the exterior surface of the pessary as depicted in partial cross section in FIG. 6. Further, the coating 54 and inner ring 56 can be flexible to allow collapsing the ring 52 for insertion in the vaginal canal where it can spring back into position in the formix area as desired. An alternative to a coating 54 on the pessary is depicted in FIG. 7. In the embodiment of FIG. 7, the surface of the ring 52 contains holes or pores 60 which are in fluid communication with an interior chamber 62 in the ring 52. The pores 60 allow elution of the vasoconstrictor over time with the pores sized for controlling elution rate while the quantity disposed in the chamber 62 controls maximum dose over time.

As previously stated, many shapes of pessaries are known in the art and could be utilized with the vasoconstrictor of the present disclosure. Further, the exterior surface of any shape can be altered in various embodiments. FIGS. 8 and 9 depict a couple alternative surface structures for a generally ring-shaped carrier member 50. In FIG. 8, the ring-shaped carrier member 50 includes undulating peaks 64 and valleys 66 which can aid in retaining the carrier member 50 within the vaginal canal and also increase the surface area for transfer of vasoconstrictor to tissue. Further, the undulating surfaces may compensate for some variation in circumference of the formix area in different individuals. FIG. 9 depicts another alternative surface configuration of undulating peaks 64 and valleys 66. The surface of the ring-shaped carrier member 50 can also include a texture that increases surface area for transfer of vasoconstrictor and also aid in retention within the vaginal canal.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

I claim:

1. A method for inhibiting uterine bleeding comprising intravaginal administration of a therapeutically effective amount of a composition comprising a vasoconstrictor to an individual in need thereof.

2. The method of claim 1 wherein the individual is asymptomatic and at risk of abnormal uterine bleeding.

3. The method of claim 1 wherein the individual has a symptom of abnormal uterine bleeding.

4. The method of claim 1 wherein the individual has been diagnosed by a medical professional with abnormal uterine bleeding.

5. The method of claim 1, wherein the composition is administered topically intravaginally and the therapeutically effective amount of composition comprises sufficient composition to effect administration of about 0.75 mg to about 3 mg vasoconstrictor.

6. The method of claim 1, wherein the vasoconstrictor is an alpha-adrenergic agonist.

7. The method of claim 5, wherein the vasoconstrictor is oxymetazoline.

8. The method of claim 1, wherein the composition further includes a pharmaceutically acceptable fluid-based carrier.

9. The method of claim 7, wherein the composition contains about 0.02% to about 0.08% oxymetazoline, and the therapeutically effective amount is about 1 cc to about 5 cc of the composition.

10. The method of claim 1, wherein the composition further includes a pharmaceutically acceptable time-release carrier.

11. The method of claim 1, wherein the vasoconstrictor is a non-hormonal compound.

12. A topical vasoconstrictor delivery system for the vaginal canal comprising: (a) a vaginally insertable tampon including an absorbent material; and, (b) a therapeutically effective amount of a composition comprising a vasoconstrictor carried by the tampon for release in the vaginal canal when the tampon is disposed therein.

13. The system of claim 12, wherein the vasoconstrictor is a non-hormonal, non-steroidal compound.

14. The system of claim 12, wherein the vasoconstrictor is an alpha-adrenergic agonist.

15. The system of claim 12, wherein the vasoconstrictor is oxymetazoline.

16. The system of claim 12, wherein the composition further includes a pharmaceutically acceptable fluid carrier.

17. The system of claim 16, wherein the tampon has an outer surface and the composition is absorbed on at least the outer surface of the tampon.

18. The system of claim 12, wherein the composition is a dry powder distributed on at least a portion of the outer surface of the tampon.

19. The system of claim 12, wherein the tampon has an outer surface and the system further comprises a carrier layer on at least a portion of the outer surface thereof having the vasoconstrictor dispersed therein for intravaginal release when the tampon is in contact with the vaginal wall.

20. The system of claim 18, wherein the carrier layer is a gel or polymer.

21. The system of claim 12, wherein the vasoconstrictor is a suppository carried proximate a distal end of the tampon.

22. A topical vasoconstrictor delivery system for the vaginal canal comprising: (a) a vaginally insertable carrier member; and, (b) a therapeutically effective amount of a composition comprising a vasoconstrictor associated with the carrier member for release in the vaginal canal when the carrier member is disposed therein.

23. The delivery system of claim 22, wherein the carrier member is a suppository operable for dissolution and in vivo absorption when placed within the vaginal canal.

24. The delivery system of claim 22, wherein the carrier member is configured and arranged as a ring, sized for positioning in a formix area, and has the vasoconstrictor associated therewith.

25. The delivery system of claim 24, wherein the composition is dispersed within a polymer carrier positioned on the outer surface of the carrier member.

26. The delivery system of claim 24, wherein the carrier member has a porous surface and the composition is disposed within the carrier member for diffusion out from the porous carrier member when the carrier member is placed intravaginally.
